(19) United States
(12) Patent Application Publication
(10) Pub. No.: US 2025/0025205 A1
(43) Pub. Date: Jan. 23, 2025

(54) FACILITATING WIRE TRACKING ACROSS ANATOMICAL STRUCTURES

(71) Applicant: Conformal Medical, Inc., Nashua, NH (US)

(72) Inventors: Aaron V. Kaplan, Norwich, VT (US); Jacob Carl Hahn, Medford, MA (US)

(21) Appl. No.: 18/775,764

(22) Filed: Jul. 17, 2024

Related U.S. Application Data
(60) Provisional application No. 63/514,305, filed on Jul. 18, 2023.

(51) Int. Cl. A61B 17/34; A61B 8/08; A61B 8/12; A61B 17/12; A61M 25/00

(52) U.S. Cl. CPC A61B 17/3403; A61B 8/0883; A61B 8/12; A61B 17/12109; A61B 17/3415; A61B 2017/1205; A61B 2017/3413; A61M 25/0068

(57) ABSTRACT

Devices and methods are described for facilitating passage of a probe or other element across an anatomical boundary. In some embodiments, the probe may be an intracardiac echo (ICE) probe, and the anatomical boundary may be the interatrial septum (IAS). The probe is delivered via transcatheter access from a femoral vein to the right atrium using a moveable guide catheter through which the probe and a wire with a resizable distal loop are advanced. The probe and wire may extend through respective lumens of the guide catheter. The loop may be resized to track, capture, and release a sheath, such that a transcatheter delivered medical device such as a left atrial appendage (LAA) occlusion device or other device, may traverse the IAS with the ICE probe via a single puncture site.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. For example, this application claims priority to the U.S. Provisional Application 63/514,305, filed Jul. 18, 2023, which is incorporated by reference herein in its entirety.

### BACKGROUND

### Field

This development relates generally to systems, devices, and methods for facilitating passage of a probe across an anatomical boundary, such as the interatrial septum (IAS). In particular, systems, devices, and methods relate to delivering an intracardiac echo (ICE) probe using a guide catheter housing the ICE probe and a wire with a retractable and resizable wire loop. A medical device, such as a left atrial appendage (LAA) occlusion device or other device, is delivered to the left atrium with the ICE probe via a single puncture site of the IAS.

### Description of the Related Art

Atrial fibrillation (Afib) is a condition in which the normal beating of the left atrium (LA) is chaotic and ineffective. The left atrial appendage (LAA) is a blind pouch off the LA. In patients with Afib blood stagnates in the LAA facilitating clot formation. These clots (or clot fragments) have a tendency to embolize or leave the LAA and enter the systemic circulation. A stroke occurs when a clot/clot fragment embolizes and occludes one of the arteries perfusing the brain. Anticoagulants, e.g., Coumadin, have been shown to significantly reduce the stroke risk in Afib patients. These drugs reduce clot formation but also increase bleeding complications including hemorrhagic strokes, subdural hematoma, and bleeding in the gastrointestinal tract.

There are about 8 million people in the US and EU with Afib. About 4.6 million of these patients are at a high risk for stroke and would benefit from anticoagulation. A large portion of these patients cannot take anticoagulants due to an increased bleeding risk, leaving their stroke risk unaddressed. The prevalence of Afib increases with age.

Several transcatheter devices for occluding the LAA to reduce stroke risk are available as an alternative to anticoagulation. These devices are metal structures which are circular in cross section and are made to expand to fill the LAA ostium. These devices are offered in many sizes and must be closely matched to the highly variable LAA anatomy, requiring precise imaging. This is difficult to do using fluoroscopy and often requires adjunctive procedural imaging in the form of transesophageal echocardiography (TEE) or Intracardiac Echo (ICE) and/or pre- and post-procedural cardiac CT and MRI, all available with three dimensional reconstructions, if necessary. Procedurally, ICE imaging is preferable over TEE as it avoids the need for general anesthesia.

While intracardiac echo (ICE) is emerging as an important modality to facilitate LAA closure, proper placement of the ICE catheter in the left atrium can be challenging. In order to provide continuous ultrasound monitoring, an ICE probe can be positioned in the LA along with the LAA sheath, which will provide access for the delivery catheter to implant the closure device in the LAA. The LA is accessed through the right atrium (RA) via a transseptal puncture (TSP) with the ICE probe in the RA. Following TSP, the ICE probe along with the LAA sheath need to be tracked across the interatrial septum (IAS). This can be accomplished by two separate TSP sites. Using two separate TSP sites can cause more risk to the patient than using one site. In some cases, both the LAA catheter and the ICE probe can access the LA via a single TSP. One method for introducing the ICE probe into the LA in this scenario can be referred to as a “shoehorn” technique.

The “shoehorn” technique starts with placement of the ICE probe in the RA using standard technique. The ICE probe is then used to guide the TSP procedure, which is typically performed using a commercially available TSP sheath/needle. After guidewire position is established across the IAS, the TSP needle is removed and the TSP sheath is exchanged for the LAA sheath, which is used to enlarge the septostomy by tracking it across the IAS. Alternatively, the LAA sheath can be used for the TSP procedure to avoid the need for a subsequent exchange. The ICE probe can then be tracked through the septostomy under fluoroscopic guidance. Positioning the ICE probe at the puncture site as well as anatomic features of the IAS can make crossing the IAS difficult using this method.

### SUMMARY

In some embodiments, a system for facilitating passage of a probe across an anatomical boundary includes a guide catheter which can house both a probe and a wire with a distal loop. The distal loop of the wire can be retractable, and the dimensions of the distal loop of the wire can also be controlled to vary the size of the wire loop. In order to house and guide both the probe and the wire, the guide catheter can include at least two lumens extending longitudinally therethrough. In some embodiments, the devices and methods herein may facilitate passage of an intracardiac echo (ICE) probe across the interatrial septum (IAS) through the same puncture site as the left atrial appendage (LAA) sheath.

In some implementations, a probe guide device for facilitating passage across an anatomical boundary, can include: a guide catheter can include a first opening on a distal end of the guide catheter; and a second opening on the distal end of the guide catheter; a probe configured to be disposed within the guide catheter, wherein the probe is longitudinally translatable relative to the guide catheter to move a distal end of the probe out of the first opening on the distal end of the guide catheter; and a retaining wire structure configured to be disposed within the guide catheter, wherein the retaining wire structure includes a looped portion at a distal end of the retaining wire structure, wherein the retaining wire structure is configured to be longitudinally translatable relative to the guide catheter to be moved out of the second opening on the distal end of the guide catheter, and wherein the probe is configured to be longitudinally translatable through the looped portion of the retaining wire structure when the looped portion of the retaining wire structure is positioned distal of the second opening on the distal end of the guide catheter.

In some implementations, the retaining wire structure is retractable. In some implementations, the probe is retractable. In some implementations, the device can include a probe catheter configured to be disposed within the guide catheter, wherein the probe catheter includes the probe. In some implementations, a width of the looped portion is adjustable from a proximal end of the retaining wire structure. In some implementations, the width of the looped portion is adjustable by partially retracting the retaining wire structure, wherein the looped portion is wider when the retaining wire structure is distally farther from the second opening on the distal end of the guide catheter.

In some implementations, the distal end of the guide catheter includes a dilator-tip extrusion. In some implementations, the guide catheter is steerable from a proximal end of the guide catheter. In some implementations, the probe catheter is steerable from a proximal end of the probe catheter. In some implementations, the looped portion is configured to expand to the inner walls of and fill a lumen of a vessel.

In some implementations, a medical device is configured to be inserted within and/or through the looped portion. In some implementations, the medical device is inserted from a different access site than the retaining wire structure. In some implementations, the medical device is inserted into a first anatomical vessel via a first access site formed in the first anatomical vessel and wherein the retaining wire structure is inserted into a second anatomical vessel via a second access site formed in the second anatomical vessel. In some implementations, the first anatomical vessel includes one of the left or right femoral veins and the second anatomical vessel includes the other of the left or right femoral veins. In some implementations, the medical device includes a left atrial appendage (LAA) closure device. In some implementations, the medical device further includes an LAA closure device disposed within an LAA closure device delivery catheter. In some implementations, a puncture needle is configured to be inserted within and/or through the looped portion.

In some implementations, the device can include a retaining wire sheath configured to be disposed within the guide catheter, and wherein the retaining wire structure is configured to be disposed within the retaining wire sheath. In some implementations, the looped portion is configured to be disengaged by retracting one end of the retaining wire structure from a proximal end of the retaining wire structure. In some implementations, the device can include a removable obturator configured to fill the first opening on the distal end of the guide catheter. In some implementations, the guide catheter is controlled robotically. In some implementations, the probe is an intracardiac echo (ICE) probe and the device is configured to facilitate access of the ICE probe and the medical device to the left atrium from the right atrium via a single transseptal puncture of the interatrial septum (IAS).

In some implementations, a method described herein for facilitating passage across an anatomical boundary can include positioning a guide catheter in an area near the anatomical boundary, wherein the guide catheter includes: a first opening on a distal end of the guide catheter; and a second opening on the distal end of the guide catheter; longitudinally translating a probe relative to the guide catheter so that a distal end of the probe extends through the first opening on the distal end of the guide catheter; longitudinally translating a retaining wire structure relative to the guide catheter so that a distal end of the retaining wire structure extends through the second opening on the distal end of the guide catheter, wherein the retaining wire structure includes a loop at a distal end of the retaining wire structure; positioning a puncture needle within the loop in the area near the anatomical boundary, wherein the puncture needle includes a puncture needle guidewire; inserting the puncture needle across the anatomical boundary into a target area; tracking the guide catheter across the anatomical boundary over the puncture needle guidewire into the target area; and inserting the probe into the target area.

In some implementations, the method can include inserting the guide catheter in a vessel and translating the guide catheter along the vessel to position the guide catheter in an area near the anatomical boundary. In some implementations, the method can include inserting the puncture needle in a vessel. In some implementations, the anatomical boundary is an interatrial septum. In some implementations, the area near the anatomical boundary is a right atrium. In some implementations, the target area is a left atrium. In some implementations, the method can include adjusting a width of the loop from a proximal end of the retaining wire structure. In some implementations, the method can include steering the guide catheter from a proximal end of the guide catheter. In some implementations, the method can include disengaging the loop by retracting a first end of a wire forming a portion of the retaining wire structure. In some implementations, the method can include removing a removable obturator from the guide catheter. In some implementations, the method can include puncturing the interatrial septum, in order to access the left atrium from the right atrium with an ICE probe and a medical device, via a single puncture site of the interatrial septum. In some implementations, the medical device includes a left atrial appendage (LAA) closure device. In some implementations, the medical device further includes a delivery catheter, at least a portion of the LAA closure device positioned within the delivery catheter when at least a portion of the medical device is advanced through the single puncture site of the interatrial septum to access the left atrium. In some implementations, the puncture needle is inserted into the area near the anatomical boundary via a first access site located in one of right or left femoral veins, and wherein the medical device is inserted into the area near the anatomical boundary via the same access site as the puncture needle. In some implementations, the guide catheter is inserted into the area near the anatomical boundary via a second access site located in the other of the right or left femoral veins.

Any of the features, components, or details of any of the arrangements or embodiments disclosed in this application, including without limitation any of the apparatus embodiments and any of the methods disclosed herein, are interchangeably combinable with any other features, components, or details of any of the arrangements or embodiments disclosed herein to form new arrangements and embodiments.

### BRIEF DESCRIPTION OF THE DRAWINGS

FIG. 1A is a top plan view of the distal tip section of an embodiment of a guide catheter.

FIG. 1B is a perspective view of the distal tip section of FIG. 1A.

FIG. 2A is a detail perspective view of the distal tip of the guide catheter of FIG. 1A, with a distal section of an ICE probe and a distal end of a lasso wire extending therefrom.

FIG. 2B is a side view of the distal of the guide catheter of FIG. 1A, with the distal end of the ICE probe and the distal end of the lasso wire extending therefrom.

FIG. 3A is a perspective view of the distal tip section of FIG. 1A, with an obturator positioned therein.

FIG. 3B is a side view of a distal portion of the guide catheter, with the obturator positioned therein.

FIG. 4A is a front view of another embodiment of a guide catheter, in a direction parallel to a longitudinal axis of the guide catheter.

FIG. 4B is a side view of the guide catheter of FIG. 4A.

FIG. 5A is a cross-sectional view of the guide catheter of FIG. 4A, taken along the line A-a of FIG. 4B.

FIG. 5B is another cross-sectional view of the guide catheter of FIG. 4A, taken along the line B-b of FIG. 4A.

FIG. 6 illustrates the guide catheter of FIG. 4A with an ICE probe inserted into one lumen and a bulbed lasso wire sheath inserted into the second lumen.

FIG. 7A is a cross-sectional view of the catheter guide of FIG. 6, taken in a plane perpendicular to the longitudinal axis of the catheter guide.

FIG. 7B is a cross-sectional view of the catheter guide of FIG. 6, taken in a plane parallel to the longitudinal axis of the catheter guide.

FIGS. 8A through 8D are cross-sectional views illustrating the deployment of a lasso wire from within a bulbed lasso wire sheath.

FIG. 8E is a cross-sectional view of the distal end of the bulbed lasso wire sheath of FIG. 8A, taken in a plane perpendicular to the longitudinal axis of the lasso wire sheath.

FIGS. 9A to 9C illustrate a process for disengaging a lasso wire from an ICE probe initially positioned within the loop of the lasso wire.

FIGS. 10A to 10V schematically illustrate a process for using a guide catheter and a lasso wire to assist in the performance of a septostomy to allow an ICE probe and other components to be guided through the puncture site.

### DETAILED DESCRIPTION OF CERTAIN EMBODIMENTS

The systems and methods described herein may facilitate passage of a probe across an anatomical boundary. In particular, the systems and methods described herein can be used to facilitate wire tracking of a medical device, instrument, or other element without a wire lumen across anatomic structures.

In some embodiments, this device comprises a guide catheter, which may alternately be referred to as a sheath. The guide catheter can be a moveable sheath. A wire may be positioned such that a portion of the wire extends through the guide catheter. The wire can be longitudinally translated through the guide catheter such that a distal portion of the wire can extend from an opening on a distal end of the guide catheter.

In certain embodiments, a guide catheter is provided which can guide both a probe and one or more wires. A wire may be positioned such that a portion of the wire extends through the guide catheter. The wire can be longitudinally translated through the guide catheter such that a distal portion of the wire can extend from an opening on a distal end of the guide catheter. In some embodiments, the guide catheter may include more than one lumen, each with a distal opening at or near the distal tip of the guide catheter, with one lumen being used to guide a probe, and another lumen being used to guide one or more wires.

In some embodiments, the wire may comprise a guidewire. In some embodiments, the wire may comprise a retaining wire such as a lasso wire with a distal loop or another suitable retention structure. The lasso wire can be used, in conjunction with a separate medical device, to guide the probe to desired locations within an anatomical region, including guiding the probe through an anatomical boundary to provide access to an anatomical region beyond the anatomical boundary.

In some embodiments, the lasso wire with a distal loop can be longitudinally translated relative to the guide catheter to adjust the dimensions of the loop, as well as to retract a distal looped portion of the retaining wire into the guide catheter. In some embodiments, the wire may comprise first and second ends located proximal of the guide catheter, with the loop formed by a section of the retaining wire or lasso wire located between the first and second ends. In some embodiments, the retaining wire or lasso wire may be biased to form a desired shape when translated out of and away from the distal end of the guide catheter. In some embodiments, a loop may be deployed to form a generally circular or ellipsoidal shape oriented generally orthogonally to a longitudinal axis of the guide catheter. The distal loop of the lasso wire can be retractable, and the dimensions of the distal loop of the lasso wire can also be controlled to vary the size of the wire loop.

In order to house and guide both the probe and the one or more wires, the guide catheter can include at least two lumens extending longitudinally therethrough. A first lumen of the two lumens can be dimensioned to receive and guide a wire, and a second lumen of the two lumens can be dimensioned to receive and guide the probe. The cross-sectional size of the probe lumen can be greater than the cross-sectional size of the wire lumen, as the loop section of a lasso wire can be compressed within the wire lumen to constrain its dimensions while within the lumen, prior to deployment. In some embodiment, a distal aperture at the end of the wire lumen can be located at a point even with or distal a distal aperture at the end of the probe lumen.

In some embodiments, the dimensions of the loop, including but not limited to a diameter of the loop, are adjustable from the proximal end of the retaining wire, such as by adjusting the positions of at least one of a first and second end of the wire. In some embodiments, the loop can be widened and narrowed from the proximal end of the guide catheter. In some embodiments, the loop can be used to define a deployment position for a procedure. The loop can serve as a retention loop.

In some embodiments, the loop can be deployed from a location at or near the distal end of the guide catheter and radially outward of a longitudinal centerline of the movable guide catheter. When deployed, the loop can be expanded to cover at least a radially inward area, including the longitudinal centerline of the guide catheter. The loop can be deployed to cover all or a substantial portion of a cross-sectional area of a vessel in which the loop is deployed. In some embodiments, the loop can be deployed to cover a cross-sectional area larger than the cross-sectional area of the guide catheter. In some embodiments, the loop can be deployed to cover a cross-sectional area including a region distal of and laterally offset from the longitudinal centerline of the guide catheter.

Deployment of the loop, and control of the size of the loop, can be used to selectively retain multiple elements at various stages in a procedure. In an embodiment where the procedure includes traversal of an anatomic structure, such as the formation and traversal of a septum in the IAS, the selective retention of various elements can facilitate the use of a probe or other instrument at multiple anatomical locations to guide the procedure. The guide catheter can also facilitate the traversal of an anatomic structure to deploy the probe at a different anatomical location during the procedure.

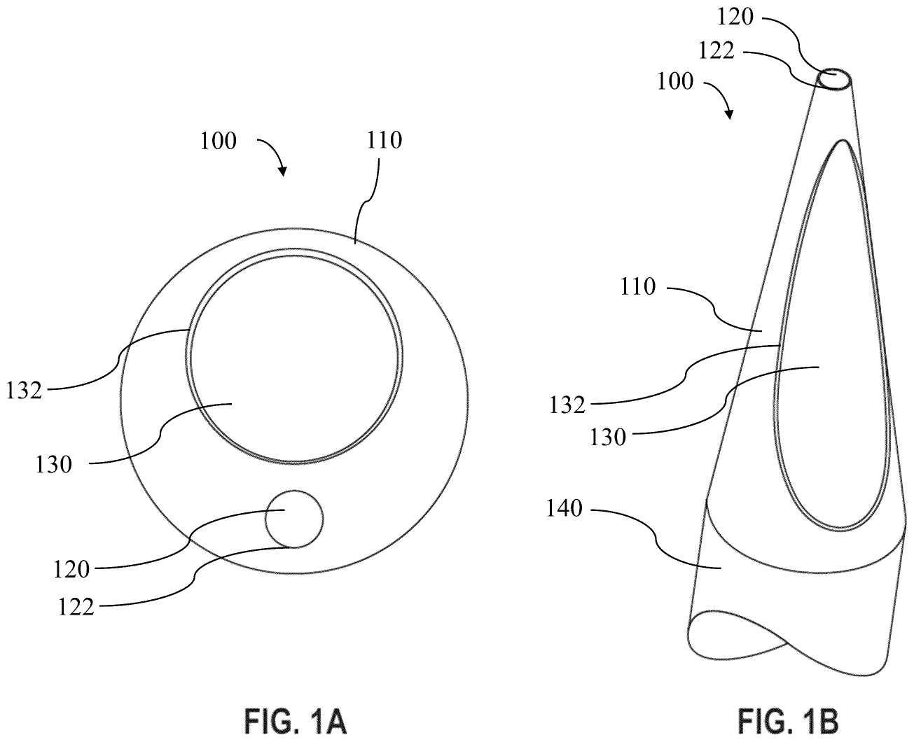

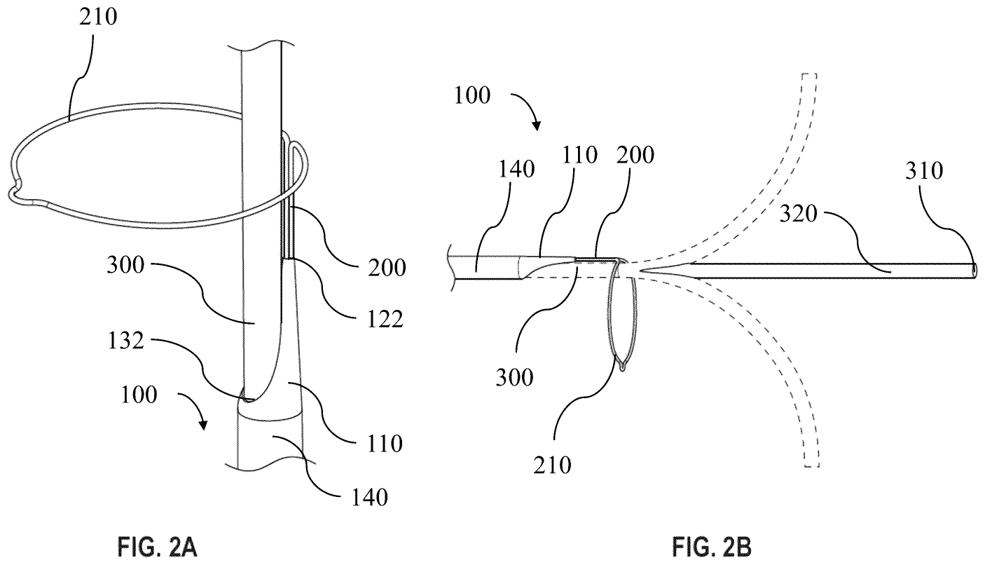

FIG. 1A is a top plan view of the distal tip section of an embodiment of a guide catheter. FIG. 1B is a perspective view of the distal tip section of FIG. 1A.

The guide catheter 100, alternatively referred to herein as a sheath, can include a tapered distal tip 110 having a generally asymmetric conical shape, whose cross-sectional size increases with distance from the distal tip 110, and a middle section 140 having a substantially constant cross-sectional shape. In the illustrated embodiment, there is a discrete transition between the distal tip 110 and the middle section 140, while in other embodiments, this transition can be more gradual. In the illustrated embodiment, the distalmost point of the distal tip 110 is laterally offset from a longitudinal centerline of the guide catheter 100. In other embodiments, however, other suitable shapes and arrangements can be used.

The guide catheter 100 can include a first lumen 120, alternatively referred to herein as a wire lumen 120, extending longitudinally through the guide catheter 100 to a distal aperture 122 at or near the distalmost portion of the distal tip 110. The guide catheter 100 can also include a second lumen 130, alternatively referred to herein as a probe lumen 130, extending longitudinally through the guide catheter 100 to a distal aperture 132 in the distal tip 110. In the illustrated embodiment, the distal aperture 132 is located in the side of the conical shape, at a location proximal the distal aperture 122 located at the tip of the conical shape.

The wire lumen 120 can have a smaller diameter than the probe lumen 130. The distal aperture 122 can have a smaller diameter than the distal aperture 132. The diameters of the probe lumen 130 and the distal aperture 132 can accommodate the diameter of the probe. The diameters of the wire lumen 120 and the distal aperture 122 can accommodate the diameter of the guidewire and/or lasso wire. In one illustrative embodiment, the wire lumen 120 may have a cross-sectional diameter of 0.037 inches, to accommodate a guidewire with a 0.035-inch diameter. In one illustrative embodiment, the probe lumen 130 may be an 11F lumen with a cross-sectional diameter of 0.144 inches to accommodate a 10F probe. In one illustrative embodiment, the guide catheter 100 may have a 17F outer cross-sectional diameter of 0.226 inches to accommodate lumens of these dimensions. Shapes and sizes of the lumens and the guide catheter 100 itself may in other embodiments be larger or smaller than these illustrative embodiments.

In some embodiments, the guide catheter 100 has an outer diameter of 1-12 mm. For example, the guide catheter 100 can have an outer diameter of about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, about 10 mm, about 10.5 mm, about 11 mm, about 11.5 mm, or about 12 mm. In some embodiments, the outer diameter of the guide catheter 100 is an outer diameter tolerated by the vasculature.

In some embodiments, the probe or a probe catheter facilitating deployment of the probe, has an outer diameter of 0.5-10 mm. For example, the probe or a probe catheter can have an outer diameter of about 0.5 mm, about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, or about 10 mm. In some embodiments, the outer diameter of the probe or a probe catheter is an outer diameter tolerated by the vasculature.

In some embodiments, the one or more distal apertures on the distal end of the guide catheter 100 have a diameter of 0.5-10 mm. For example, the one or more distal apertures on the distal end of the guide catheter 100 have a diameter of about 0.5 mm, about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, or about 10 mm. In some embodiments, the distal aperture 132 for the probe lumen 130 has a larger diameter than the distal aperture 122 for the wire lumen 120. In some embodiments, the opening for a probe catheter lumen has a larger diameter than the distal aperture 122 for the wire lumen 120.

In some embodiments, the distal tip 110 of the guide catheter 100 is a dilator-tip extrusion. In some embodiments, the dilator-tip is flexible. In some embodiments, the guide catheter 100 comprises an obturator configured to smooth the dilator tip. Advantageously, this can allow for easier septal crossing. In some embodiments, the obturator is removable.

In some embodiments, the loop is configured to fill a lumen of a vessel. In some embodiments, at least one medical tool is configured to be inserted through the loop and selectively retained by the loop, such as by controlling the dimensions of the loop. At least one medical tool to be retained by the retaining wire can be inserted from a different access site than the access site used to insert the retaining wire. In some embodiments, a puncture needle is configured to be inserted through the loop and selectively retained by the loop. In some embodiments, a guidewire is configured to be inserted through the loop and selectively retained by the loop. In some embodiments, a puncture needle guidewire is configured to be inserted through the loop and selectively retained by the loop. In some embodiments, a component inserted from another access site can be inserted through the loop and selectively retained by the loop. In some embodiments, the loop can be narrowed before the wire is inserted in an opening in a boundary. In some embodiments, the loop can be disengaged while a medical tool or other structure extends through the loop, such as by retracting a first end of the retaining wire on the proximal end of the retaining wire, in order to pull the second end of the retaining wire around the medical tool and subsequently out of the guide catheter. In some embodiments, the retaining wire has a bias that causes the loop to form when part of the retaining wire is not within the guide catheter.

In some embodiments, a probe is located at least partially within the guide catheter 100. In some embodiments, the probe can be within a probe catheter, or the probe can be part of a probe catheter. In some embodiments, the probe traverses within a probe catheter. In some embodiments, the probe is an ICE probe. In some embodiments, the guide catheter 100 has one opening on the distal end of the guide catheter 100 for the wire and the probe. In some embodiments, the guide catheter 100 has two openings on the distal end of the guide catheter 100 so that the probe can move out of a first opening and the wire can move out of a second opening. The probe and/or the probe catheter can be longitudinally translated related to the guide catheter 100 such that the probe extends through an opening on the distal end of the guide catheter 100. In some embodiments, the wire can extend through an opening on the distal end of the guide catheter 100. In some embodiments, the probe catheter can be steered from the proximal end of the probe catheter. In some embodiments, the probe catheter can be steered robotically. Advantageously, the loop can surround a portion of the probe catheter, such a portion proximal a distal section of the probe catheter without substantially impeding the operation and/or movement of the probe catheter.

In some embodiments, the guide catheter 100 can be referred to as an ICE Guide. In some embodiments, the probe can be referred to as an ICE Probe. In some embodiments, the probe catheter can be referred to as an ICE Probe. In some embodiments, the wire can be referred to as the lasso wire. In some embodiments, the wire can be referred to as the Halo wire. In some embodiments, the wire can be referred to as the lasso guidewire. In some embodiments, the wire can be referred to as the Halo guidewire.

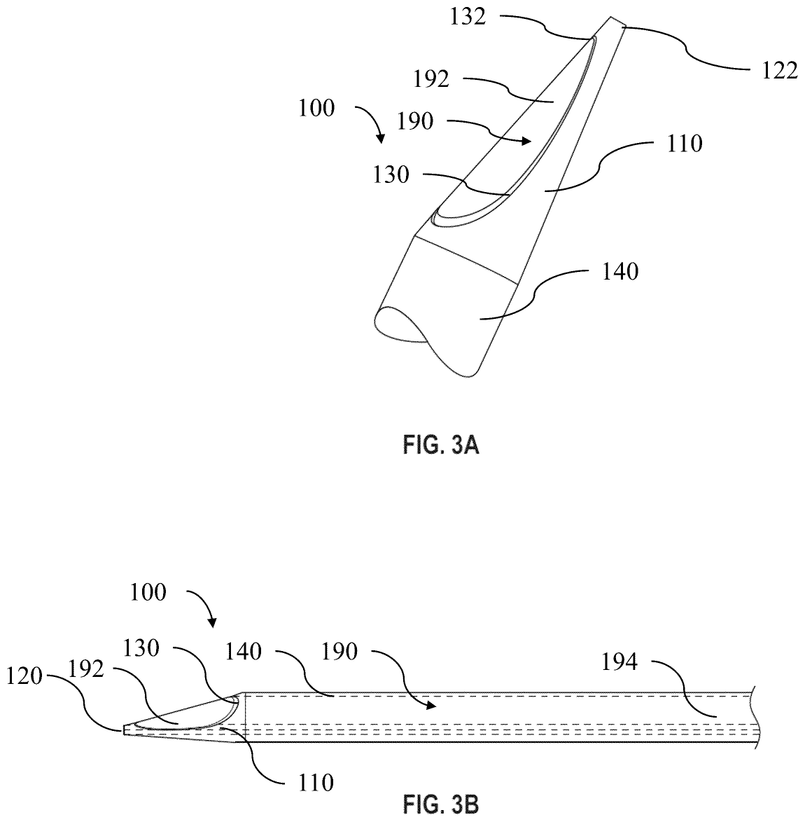

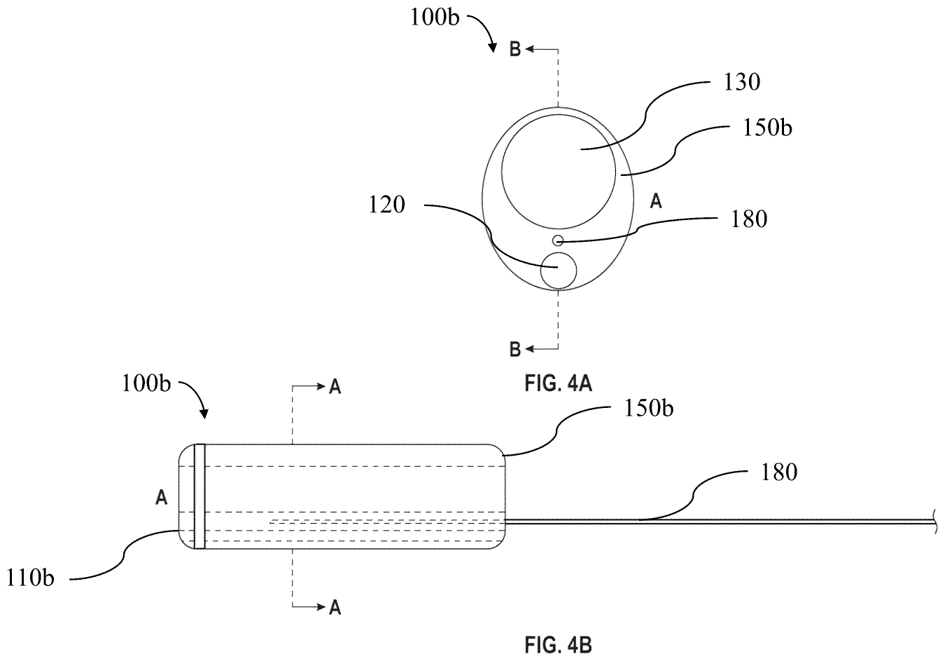

FIG. 2A is a detail perspective view of the distal tip of the guide catheter of FIG. 1A, with a distal section of an ICE probe and a distal end of a lasso wire extending therefrom. In the illustrated embodiment, the distal end of the lasso wire 200, including a loop 210, extends from the distal aperture 122 at the distalmost portion of the distal tip 110. The ICE probe 300 extends through the loop 210 at the distal end of the lasso wire 200.

FIG. 2B is a side view of the distal of the guide catheter of FIG. 1A, with the distal end of the ICE probe and the distal end of the lasso wire extending therefrom. As shown via the dashed lines, the section of the ICE probe 300 adjacent the distal tip 310 of the ICE probe 300 is a steerable section 320 extending beyond the loop 210 of the lasso wire 200, allowing the position of the distal tip 310 of the ICE probe to be directly controlled over a lateral range which is comparatively larger than the diameter of the distal tip 110 of the guide catheter 100.

The ICE probe 300 can have a working length, or a length that may be advanced through the guide catheter 100 and/or beyond the distal tip 110 of the guide catheter 100. In some embodiments, the working length of the ICE probe 300 is a length tolerated by the vasculature. In some embodiments, the working length of the ICE probe 300 is between approximately 75 cm and 78 cm. In some embodiments, the working length of the ICE probe 300 is between approximately 60 cm and 90 cm. In some embodiments, the working length of the ICE probe 300 is between approximately 50 cm and 100 cm.

In some embodiments, the steerable section of the ICE probe 300 is approximately 7.5 cm. In some embodiments, the steerable section of the ICE probe 300 is between approximately 5 cm and 10 cm. The ICE probe 300 can have a hub length of between approximately 2 cm and 5 cm. The ICE probe 300 can have a hub length of between approximately 1 cm and 8 cm. The ICE probe 300 can protrude approximately 10 cm from the distal end of the guide catheter 100. The ICE probe 300 can protrude between approximately 5 cm and 15 cm from the distal end of the guide catheter 100.

These dimensions are representative of a single illustrative embodiment, and in no way constrain the dimensions or shapes of the various components of an embodiment of a guide catheter 100 according to the disclosed technology. In particular, when embodiments of a guide catheter are used to facilitate crossing of or tracking across another type of anatomical boundary, the guide catheter 100 and other components may be dimensioned as appropriate for those other usages of the disclosed technology.

In some embodiments, a removable obturator can be used to smooth the outer profile of the distal tip 110 of the guide catheter 100 at certain points in its usage. For example, the use of a removable obturator can provide a smoother shape of the distal tip 110 of the guide catheter 100 during septal crossing, when the guide catheter 100 is being advanced through a septum.

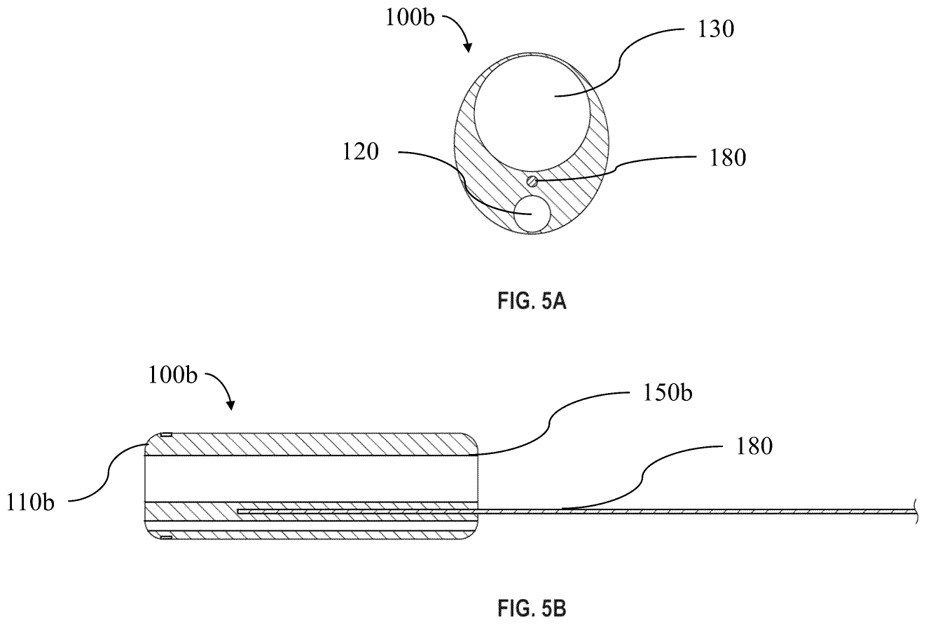

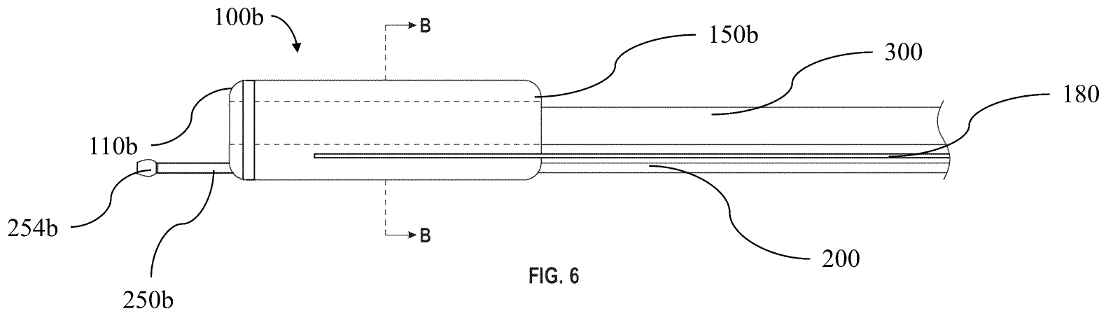

FIG. 3A is a perspective view of the distal tip section of FIG. 1A, with an obturator positioned therein. FIG. 3B is a side view of a distal portion of the guide catheter, with the obturator positioned therein.

In the illustrated embodiment, the distal tip 192 of the obturator 190 has an asymmetrical shape which matches the asymmetrical conical profile of the distal tip 110 of the guide catheter 100. As can be seen in FIG. 3B, the cross-sectional shape of the cylindrical proximal portion 194 of the obturator 190 in the illustrated embodiment has a cylindrical shape with a diameter close to that of the second lumen 130 of the guide catheter 100. In other embodiments, however, the various sections of the obturator 190 can have other shapes and sizes.

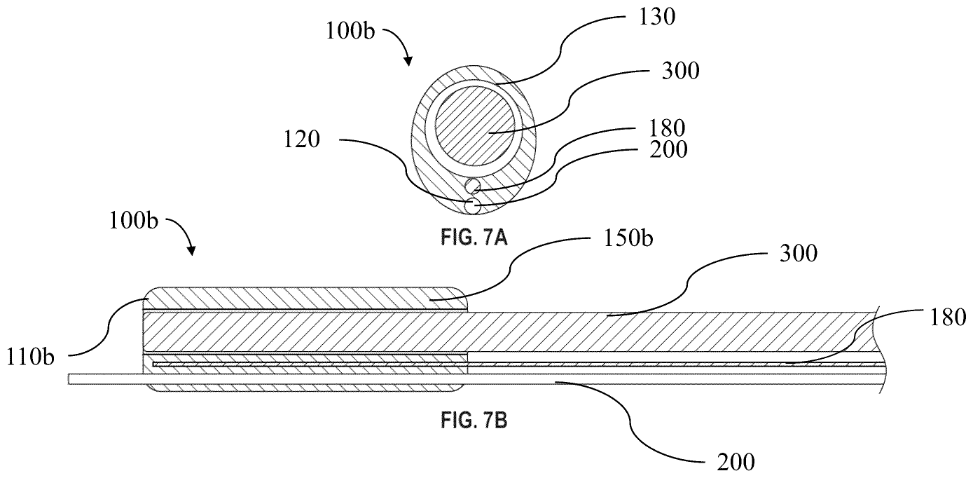

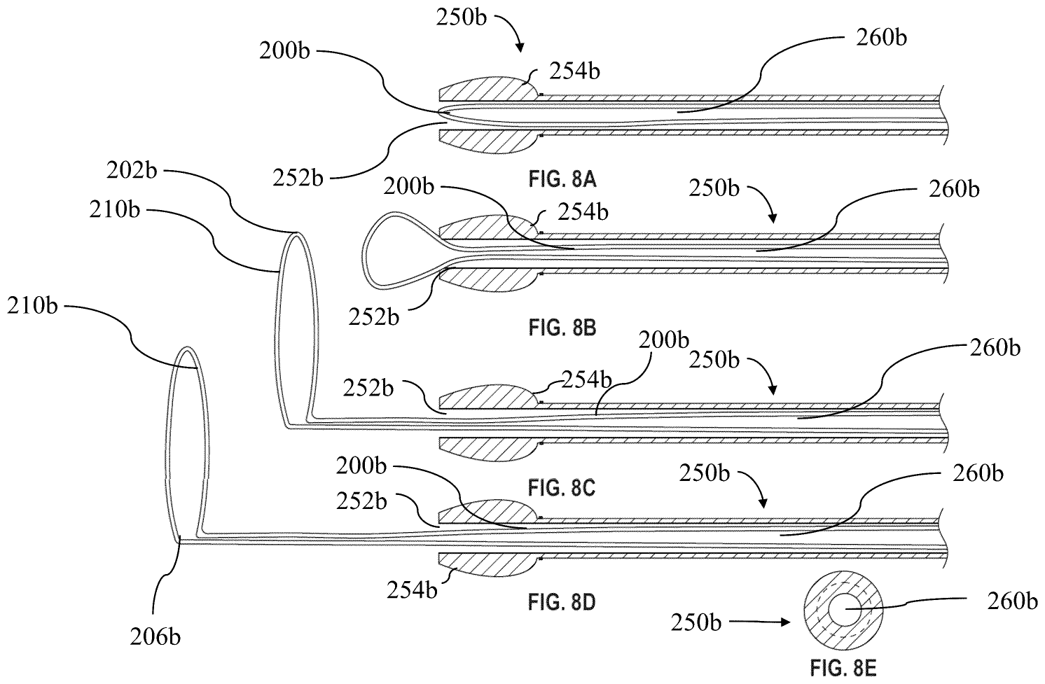

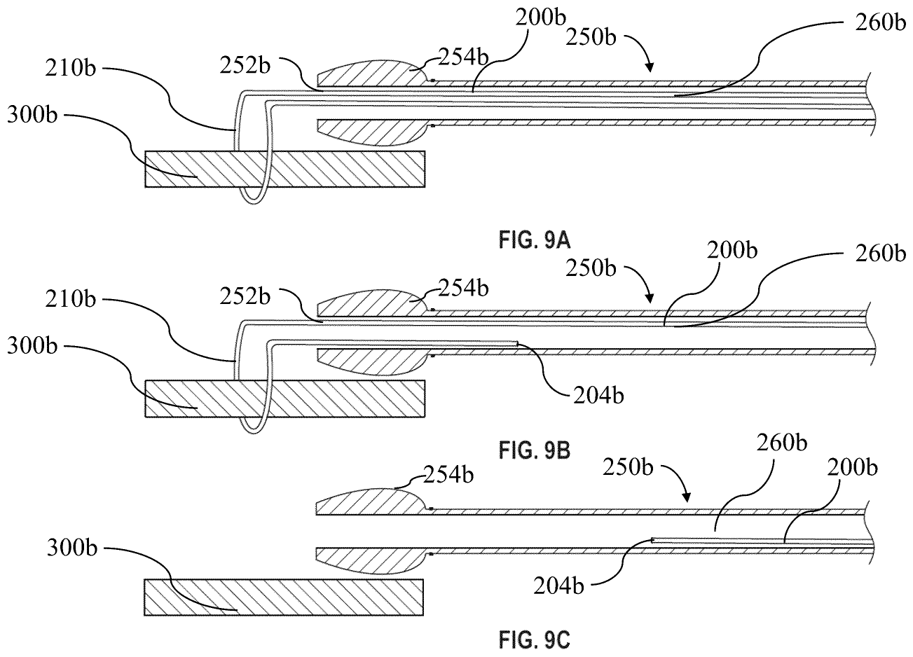

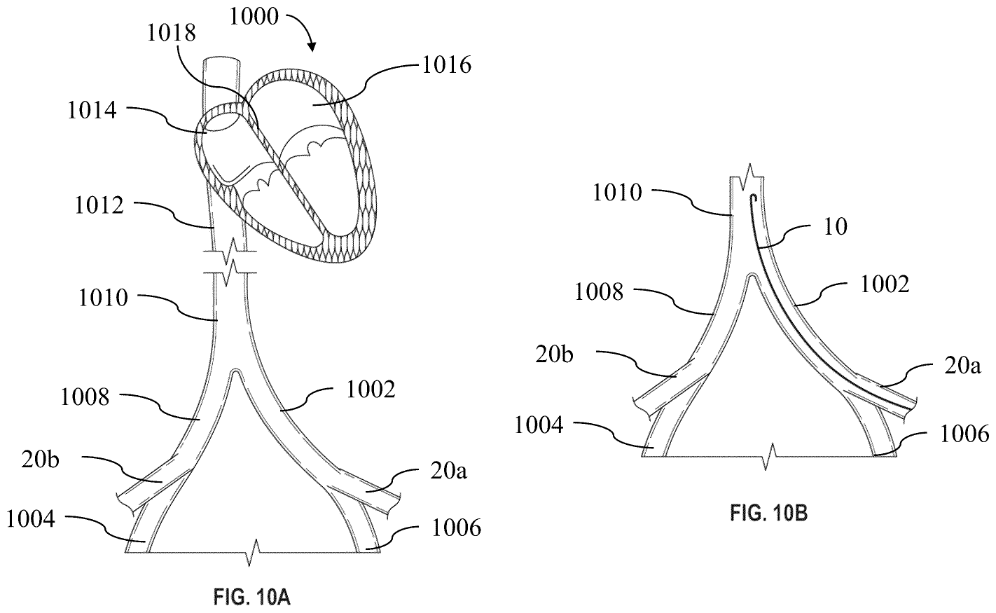

FIG. 4A is a front view of another embodiment of a guide catheter, in a direction parallel to a longitudinal axis of the guide catheter. FIG. 4B is a side view of the guide catheter of FIG. 4A. FIG. 5A is a cross-sectional view of the guide catheter of FIG. 4A, taken along the line A-a of FIG. 4B. FIG. 5B is another cross-sectional view of the guide catheter of FIG. 4A, taken along the line B-b of FIG. 4A.

The guide catheter 100b of FIG. 4A differs from the guide catheter 100 of FIG. 1A in that the guide catheter 100b can be a comparatively shorter structure having a thinner push rod 180 extending from the proximal end 150b of the guide catheter 100b. The guide catheter 100b in the illustrated embodiment has a generally cylindrical shape with rounded corners at the distal end 110b and the proximal end 150b, in contrast to the more conical distal tip 110 of the guide catheter 100 of FIG. 1A. The cross-sectional shape of the guide catheter 100b is somewhat elliptical, in comparison to the circular cross-section of the middle section 140 of the guide catheter 100 of FIG. 1A.

In some embodiments, the retaining wire 200 is positioned within and extends through a retaining wire sheath, which can have an opening on its distal end. In some embodiments, the retaining wire sheath extends through at least a portion of the guide catheter 100 and can be longitudinally translated relative to the guide catheter 100. The retaining wire sheath can be translated out of an opening on a distal end of the guide catheter 100. The retaining wire sheath can have a wider bulb portion on the distal end. In some embodiments, the retaining wire sheath can keep the retaining wire loop 210 from expanding while it is in the retaining wire sheath and can be used to provide additional control over a deployment location of a loop 210 of the retaining wire 200, by moving the retaining wire sheath prior to or after translation of the retaining wire loop 210 out of the distal end of the retaining wire sheath. In some embodiments, the retaining wire 200 has a bias that causes the loop 210 to form when a distal section of the retaining wire 200 is not within the retaining wire sheath.

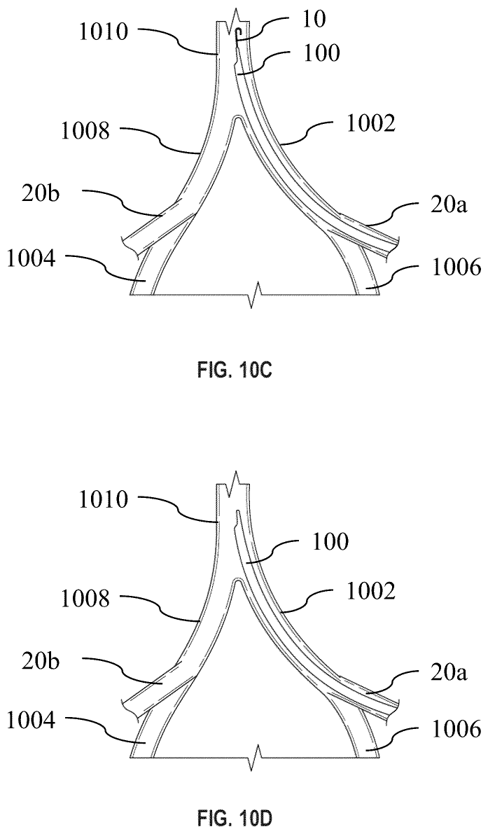

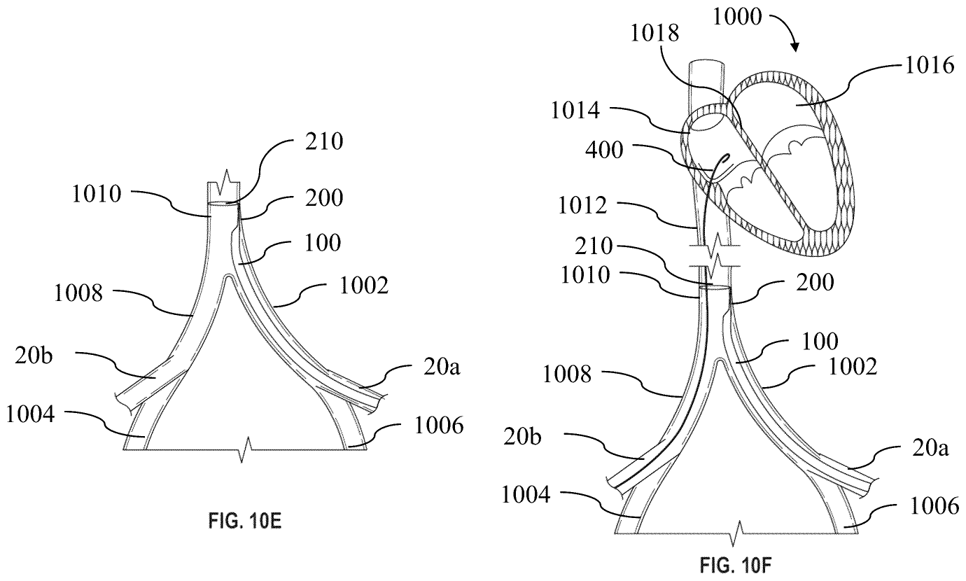

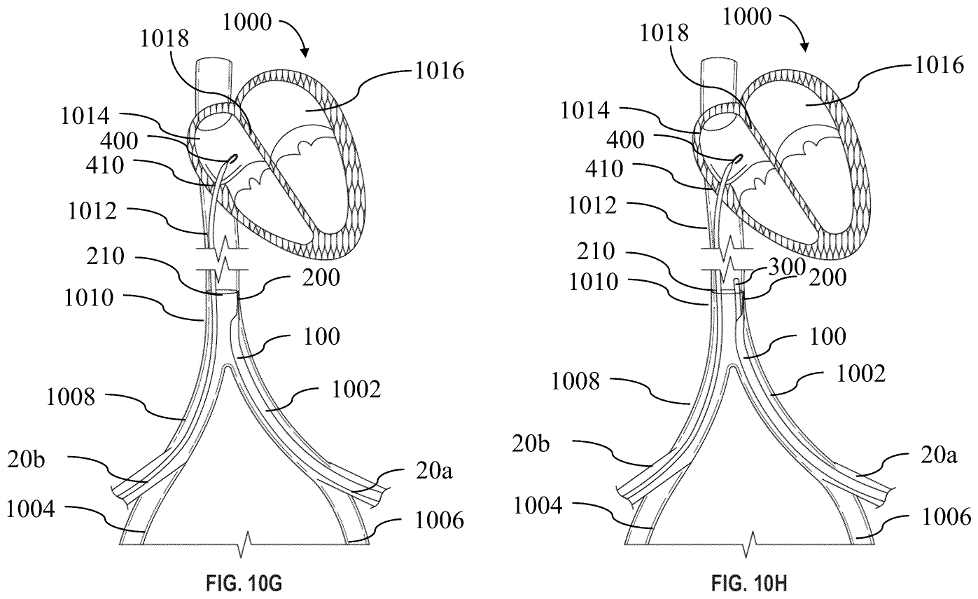

FIG. 6 illustrates the guide catheter of FIG. 4A with an ICE probe inserted into one lumen and a bulbed lasso wire sheath inserted into the second lumen. FIG. 7A is a cross-sectional view of the catheter guide of FIG. 6, taken in a plane perpendicular to the longitudinal axis of the catheter guide. FIG. 7B is a cross-sectional view of the catheter guide of FIG. 6, taken in a plane parallel to the longitudinal axis of the catheter guide.

The retaining wire sheath 250b can have a bulb 254b on the distal end of the retaining wire sheath 250b. In some embodiments, the loop 210 is configured to narrow as the lasso wire 200 is retracted to reduce an amount of the wire outside the opening on the distal end of the guide catheter 100 and retaining wire sheath 250b. In some embodiments, the amount of wire outside the opening on the distal end of the wire sheath 250b can be controlled from the proximal end of the wire sheath 250b. In some embodiments, the amount of wire outside the opening on the distal end of the guide catheter 100 can be controlled from the proximal end of the guide catheter 100. In some embodiments, the wire can be disengaged by retracting one end of the wire. This can be advantageous, as the wire can be retracted even when the wire surrounds a separate medical tool.

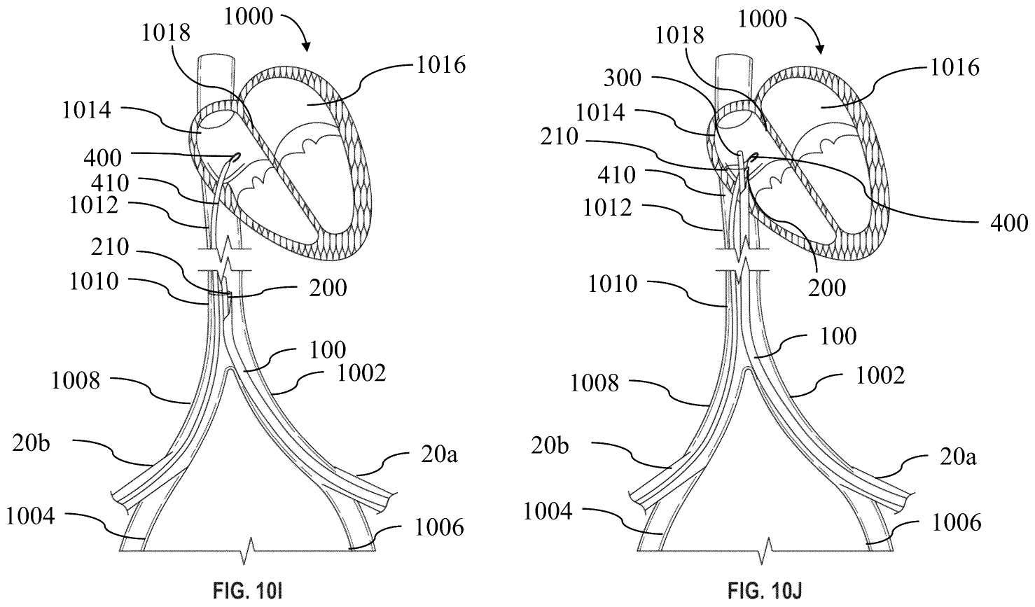

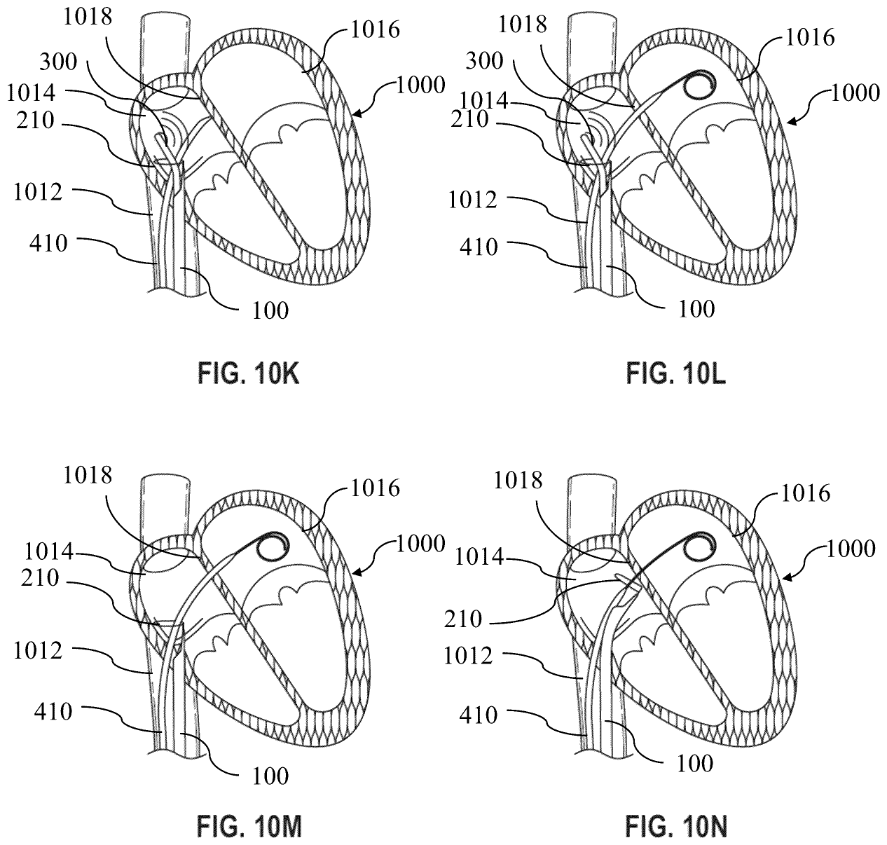

As shown in FIGS. 8A to 8D, the wire can be advanced through the central lumen of the retaining wire sheath 250 to form a loop 210. The loop 210 can surround a targeted object 300b. The loop 210 can then be tightened by retracting one or both ends of the wire. Tightening the loop 210 around the target object 300b can more readily allow the object to be manipulated.

In some embodiments, the proximal end of the guide catheter 100 can be outside the patient. In some embodiments, the proximal end of the guide catheter 100 can have a hub. In some embodiments, the proximal end of the guide catheter 100 can have a valve. In some embodiments, the valve can have a sidearm. In some embodiments, the proximal end of the guide catheter 100 can have a port. In some embodiments, the proximal end of the guide catheter 100 can have a port for the wire 200. In some embodiments, the proximal end of the guide catheter 100 can have a port for the probe 300. In some embodiments, the proximal end of the guide catheter 100 can have a port for the probe catheter. In some embodiments, the proximal end of the guide catheter 100 can have a port for a guidewire. In some embodiments, the same port used for a wire 200 or a wire sheath can be used for a guidewire.

FIGS. 8A through 8E are cross-sectional views illustrating the deployment of a lasso wire from within a bulbed lasso wire sheath. FIG. 8E is a front view of the distal end of the bulbed lasso wire sheath of FIG. 8A. As can be seen in FIG. 8A, the lasso wire 200b does not extend out of the wire lumen 260b within the wire sheath 250b. The lasso wire 200b is bent at its distal tip 202b, with both ends of the wire 200b extending towards the proximal end of the wire sheath 250b.

By advancing at least one of the ends of the wire 200b in a distal direction, the loop 210b at the distal end of the lasso wire 200b is pushed out of the distal aperture 252b in the bulbed distal end 254b of the wire sheath 250b. The distalmost section of the wire 200b may form or be biased to form a rounded loop when it is moved beyond the constraints of the wire lumen 260b, as can be seen in FIG. 8B. In addition, the wire 200b may bend or be biased to bend at points 206b along the wire, such that when a sufficient portion of the wire 200b is pushed out of the distal aperture 252b and the points 206b at the ends of the loop 210b are located outside of the constraints of the wire lumen 260b, the loop 210b is bent into an orientation where it is generally perpendicular to the remainder of the wire 200b and the longitudinal axis of the wire sheath 250b.

Further extension of the wire 200b out of the wire lumen 260b moves the loop 210b further from the bulbed distal end 254b of the wire sheath 250b while maintaining the orientation of the loop 210b. In certain embodiments, the wire 200b may be dimensioned such that the loop 210b will extend to roughly the cross-sectional diameter of a vessel or other body cavity into which it is designed to be inserted and expanded.

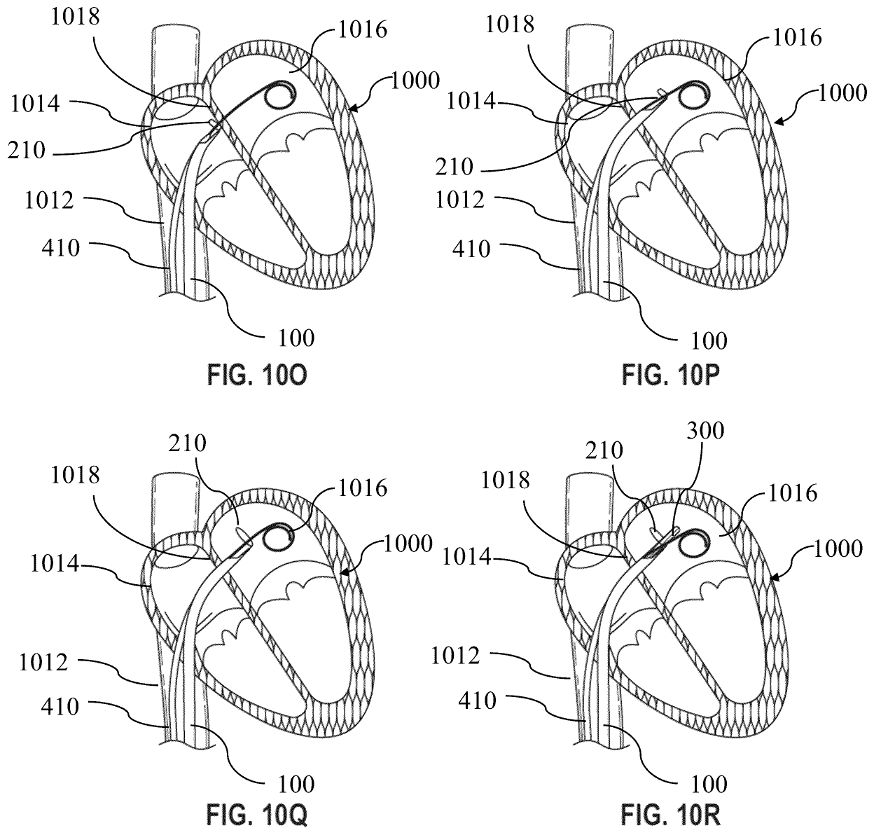

FIGS. 9A to 9C illustrate a process for disengaging a lasso wire from an ICE probe initially positioned within the loop of the lasso wire. In FIG. 9A, the ICE probe 300 is initially positioned such that it extends through the loop 210b at the distal end of the wire 200b. In FIG. 9B, one end of the wire 200b has been retracted, pulling the other end 204b in a distal direction toward the loop 210b. With further retraction, the end 204b of the wire 200b has been pulled along the loop 210b, and then retracted back into the wire lumen 260b, to disengage the wire 200b from the ICE probe 300b without the need to move any other components of the ICE probe 300, the wire sheath 250b, or a guide catheter through which the ICE probe 300 and the wire sheath 250b extend.

In some embodiments, the devices and methods described herein can facilitate easier transport across a boundary, such as a boundary in a heart or a boundary elsewhere in a vasculature. In some embodiments, the loop on the distal end of the lasso wire 200 can be narrowed to fit through an opening. In some embodiments, the loop 210 on the distal end of the lasso wire 200 can be narrowed to stabilize movement of tools within the loop 210, and/or well as to secure multiple tools together. In some embodiments, the loop 210 on the distal end of the lasso wire 200 can be widened to allow a greater degree of movement of tools within the loop 210. In some embodiments, the loop 210 can be widened to maintain a width of a lumen, or to enlarge a width of a lumen.

In some embodiments, a method for facilitating passage across a boundary includes inserting a guide catheter 100 in a vessel. In some embodiments, the method for facilitating passage across a boundary includes inserting a puncture needle 410 in the vessel. In some embodiments, the puncture needle 410 includes a puncture needle guidewire 400. In some embodiments, the puncture needle 410 moves through a loop 210 on a retaining wire. In some embodiments, the puncture needle 410 moves across a boundary into a target area. In some embodiments, the probe 300 can be positioned in the target area. In some embodiments, the probe catheter can be positioned in the target area.

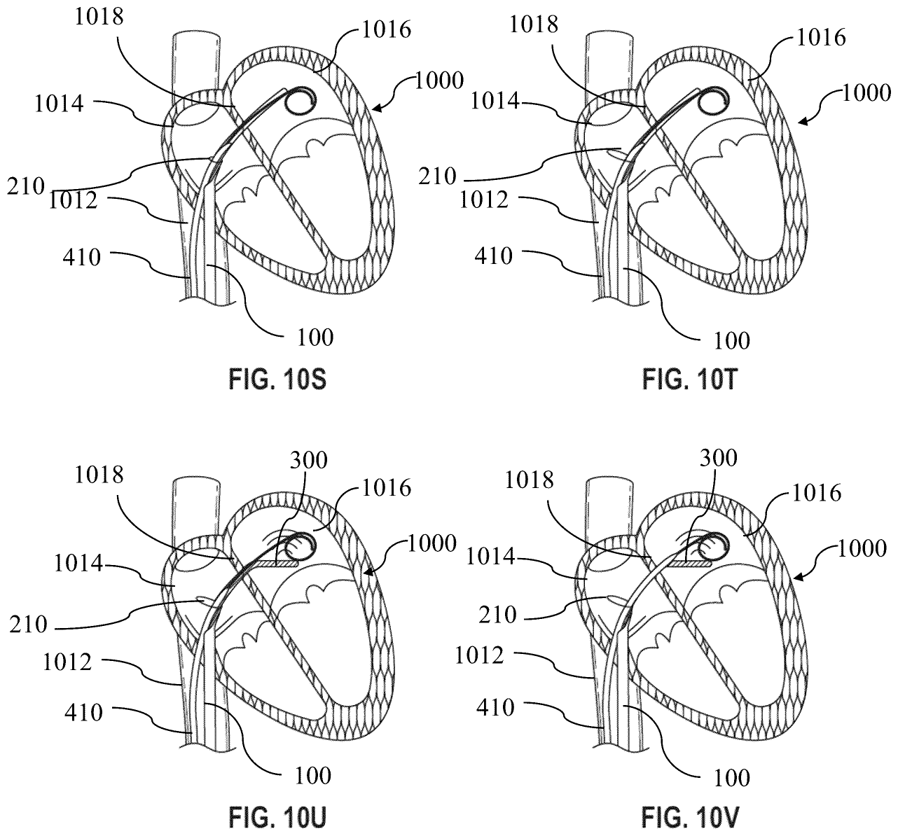

FIGS. 10A to 10V schematically illustrate a process for using a guide catheter and a lasso wire to assist in the performance of a septostomy to allow an ICE probe and other components to be guided through the puncture site. FIGS. 10C to 10V depict the guide catheter 100 of FIG. 1A, but in other implementations a catheter such as the guide catheter 100b of FIG. 4A, or any other embodiment of a guide catheter, can also be used.

As shown in FIG. 10A, the heart 1000 and the surrounding vasculature can be the site of the septostomy. The left femoral vein 1002 can lead to the left common iliac vein 1006. The right femoral vein 1004 can lead to the right common iliac vein 1008. The left common iliac vein 1006 and the right common iliac vein 1008 can join at the common iliac vein 1010. The common iliac vein 1010 can lead to the inferior vena cava 1012. The inferior vena cava 1012 can lead to the right atrium 1014 of the heart 1000. The right atrium 1014 can be adjacent to the left atrium 1016, separated by the interatrial septum 1018.

In some embodiments of the methods described herein, bilateral venous access is obtained via the right and left femoral veins 1002, 1004. In some embodiments, short introducer sheaths 20a and 20b can be inserted in each of the right and left femoral veins 1002, 1004. The introducer sheath 20a introduced to the right femoral vein 1004 can be used to introduce a transseptal puncture apparatus. In some embodiments, the introducer sheath 20b introduced to the right femoral vein 1004 can be used to introduce a left atrial appendage guide catheter. The introducer sheath introduced to the left femoral vein 1002 can be for a probe. In some embodiments, the probe is an ICE probe. In some embodiments, a sheath introduced to the left femoral vein 1002 is for a probe guide device described herein. In other embodiments, both punctures can occur in the left femoral vein 1002. In other embodiments, both punctures can occur in the right femoral vein 1004.

As shown in FIG. 10B, a guidewire 10 can be inserted into a vessel using an introducer sheath. In some embodiments, the guidewire 10 can be a puncture needle guidewire. In some embodiments, the guidewire 10 can be another guidewire suitable for positioning in the vasculature. In some embodiments, a guidewire 10 can be inserted through the left femoral vein 1002 to a point beyond the convergence of the left common iliac vein 1006 and the right common iliac vein 1008 at the common iliac vein 1010 and in some embodiments up to the lower inferior vena cava (IVC) 1012.

As shown in FIG. 10C, a guide catheter 100 can also be inserted in a vessel. In some embodiments, the guide catheter 100 is inserted in the left femoral vein 1002 over the guidewire 10 to a point beyond the convergence of the left common iliac vein 1006 and the right common iliac vein 1008 at the common iliac vein 1010 and in some embodiments up to the lower inferior vena cava (IVC) 1012. The guide catheter 100 can have at least one opening on the distal end of the guide catheter 100, as described herein. In some embodiments, the guide catheter 100 can be a multi-lumen guide catheter with at least two openings on the distal end of the guide catheter 100. A wire lumen of the guide catheter 100 can be inserted over the guidewire 10, leaving the other lumen of the guide catheter 100 unobstructed. In some embodiments, the guide catheter 100 may have a sufficient length that a portion of the guide catheter 100 extends from the insertion site, and advancement of the guide catheter 100 can be performed by moving a proximal portion of the guide catheter 100. In some embodiments, the guide catheter 100 can include a push rod 180 at a proximal end of the guide catheter 100, and advancement of the guide catheter 100 can be performed by moving a proximal portion of the push rod 180.

As shown in FIG. 10D, the guidewire 10 can be retracted from the left femoral vein 1002 after the guide catheter 100 has been inserted, leaving the guide catheter 100 in place. At this stage, both lumens of the guide catheter 100 are free to receive components inserted from the proximal end (not shown) of the guide catheter 100.

As shown in FIG. 10E, a retaining wire, or lasso wire 200, is inserted through the guide catheter 100 within the left femoral vein 1002. In some embodiments, the lasso wire 200 has a loop 210 on the distal end of the lasso wire 200. In some embodiments, the lasso wire 200 is inserted sufficiently far that the lasso wire 200 extends out of the opening on the distal end of the guide catheter 100 to deploy the loop 210 at the distal end of the lasso wire 200. In some embodiments, the loop 210 can contact a lumen such as the lumen of the common iliac vein 1010 or the IVC 1012. In some embodiments, the loop 210 can expand to encompass most of the lumen or the entire lumen, such as the entire common iliac vein 1010 or IVC 1012 lumen.

As shown in FIG. 10F, a puncture needle guidewire 400 can be inserted in the right femoral vein 1004 and advanced through the loop 210 in the lasso wire 200. As shown, the puncture needle guidewire 400 can be advanced through the loop 210 and through the inferior vena cava 1012 into the right atrium 1014 of the heart 1000. In some embodiments, the puncture needle guidewire 400 is a guidewire configured to be disposed within a puncture needle. The puncture needle guidewire 400 can pierce a heart wall and enter an area of the heart, such as the right atrium 1014.

As shown in FIG. 10G, a puncture needle 410 can be inserted in the right femoral vein 1004 over the puncture needle guidewire 400. The puncture needle 410 can be a guidewire sheath. In some embodiments, the puncture needle 410 is a transseptal access sheath or delivery system used for transseptal intervention. In some embodiments, the puncture needle 410 is a transseptal puncture (TSP). The puncture needle 410 can have an opening on its distal end. As shown, the puncture needle 410 can be advanced along the puncture needle guidewire 400 through the loop 210 and through the inferior vena cava 1012 into the right atrium 1014 of the heart 1000. In the state illustrated in FIG. 10G, an instrument in the form of a puncture needle 410 inserted via a first access point in a first vessel extends through a loop 210 in a lasso wire 200 inserted via a second access point in a second vessel.

As shown in FIG. 10H, the probe 300 can be positioned within the open lumen of the guide catheter 100 and advanced such that it extends through at least a portion of the guide catheter 100 within the left femoral vein 1002. The probe 300 may be within a probe catheter or may be part of a probe catheter. In some embodiments, the probe 300 can traverse along the probe catheter. The probe 300 extends through one lumen of the guide catheter 100, while the lasso wire can extend through a second, separate lumen of the guide catheter 100.

Although the probe 300 in the illustrated embodiment is depicted as being inserted and advanced after the puncture needle 410 and the puncture needle guidewire 400, the probe 300 can in other embodiments be inserted and advanced through the loop 210 of the lasso wire 200 prior to insertion and advancement of the puncture needle guidewire 400 and the puncture needle 410.

As shown in FIG. 10I, the loop 210 at the distal end of the lasso wire 200 can be narrowed around the probe and the puncture needle. The narrowing of the loop 210 reduces the cross-sectional size of the loop in a direction orthogonal to the surrounding vessel to a size less than the diameter of the surrounding vessel. In some embodiments, one or both ends of the lasso wire 200 can extend from the proximal end of the guide catheter 100, and the loop 210 can be narrowed by retracting one or both ends of the lasso wire 200 in a proximal direction away from the proximal end of the guide catheter 100.

As shown in FIG. 10J, the guide catheter 100, the lasso wire 200, and the probe 300 can be advanced into the right atrium 1014 along the puncture needle 410. A length of the distal end of the probe 300 extends beyond the loop 210 at the distal end of the lasso wire 200. In some embodiments, some or all of this length of the distal end of the probe 300 extending beyond the loop 210 can be steerable to maneuver the tip of the probe 300 within the right atrium 1014 or another anatomical region into which the probe 300 has been advanced.

As shown in FIG. 10K, the probe 300 can be used to guide the puncture needle 410, such as by using ultrasound or echocardiogram. In some embodiments, the probe 300 can be used to determine how to puncture the boundary with the puncture needle 410. In some embodiments, the probe 300 can be used to determine how to puncture the interatrial septum (IAS) 1018 with the puncture needle 410. In some embodiments, the information provided by the probe 300 can be used to direct the puncture needle 410 to the location at which the septectomy is to be performed. In some embodiments, the puncture needle 410 can puncture the boundary without guidance from the probe 300.

In the illustrated embodiment, the distal ends of the guide catheter 100 and the lasso wire 200 are depicted as extending into the right atrium 1014. However, in other embodiments, the distal ends of the guide catheter 100 and the lasso wire 200 may at this stage be positioned slightly outside of the right atrium 1014, in the IVC 1012. For example, if the steerable section at the distal end of the probe 300 is sufficiently long and/or movable, sufficient information can be provided to direct the puncture needle 410 without advancing the guide catheter 100 and the lasso wire 200 into the right atrium 1014 at this stage.

As shown in FIG. 10L, the puncture needle 410 can be inserted across the IAS 1018 and advanced into the left atrium 1016. This advancement of a puncture needle 410 inserted via a first access point in a first vessel to perform an atrial septostomy is performed while at least a portion of the puncture needle 410 extends through a loop 210 in a lasso wire 200 inserted via a second access point in a second vessel. This septostomy can also be performed while the same loop 210 retains a portion of a probe 300 also inserted via the second access point in the second vessel.

As shown in FIG. 10M, the probe 300 can be retracted at least partially within the guide catheter 100. The retraction of the probe 300 can facilitate the traversal of the guide catheter 100 through the septum. In some embodiments, an obturator can be inserted via the lumen of the guide catheter 100 used for the probe 300 to smooth the tip of the guide catheter 100. Advantageously, this can allow for easier septal crossing.

As shown in FIG. 10N, the puncture needle 410 can be retracted from the left atrium 1016. The puncture needle guidewire 400 can remain in place, extending through the septum into the left atrium 1016. While the puncture needle 410 can be retracted to a position proximal the loop 210 of the lasso wire 200, the puncture needle guidewire 400 still extends through the loop 210 of the lasso wire 200.

As shown in FIG. 10O, the method can include narrowing the loop 210 on the distal end of the lasso wire 200 around the puncture needle guidewire 400. Because neither the probe 300 nor the puncture needle 410 extend through the loop 210 of the lasso wire 200 at this stage, the loop 210 can be further narrowed to facilitate the passage of the loop 210 and the guide catheter 100 through the septum into the target area in the left atrium 1016.

As shown in FIG. 10P, the guide catheter 100 can be tracked across the boundary over the puncture needle guidewire 400 into the target area. The guide catheter 100 can be tracked across the IAS 1018 over the puncture needle guidewire 400 into the left atrium 1016. Upon traversal of the guide catheter 100 into the right atrium 1016, the distal opening at the distal end of the probe lumen of the guide catheter 100 is located within the right atrium 1016. In an embodiment in which an obturator was positioned within the probe lumen of the guide catheter 100 during traversal of the guide catheter 100 across the IAS 1018, the obturator can now be removed.

As shown in FIG. 10Q, the method can include widening the loop 210 around the puncture needle guidewire 400. In an embodiment in which one or both ends of the lasso wire 200 extend from the proximal end of the guide catheter 100, the loop 210 can be widened by advancing one or both of the ends of the lasso wire 200 in a distal direction toward the distal end of the guide catheter 100.

As shown in FIG. 10R, the probe 300 can be inserted through the opening on the distal end of the guide catheter 100. The probe 300 can be inserted through the loop 210. Because the wire loop 210 has been widened, the probe 300 can more readily be inserted through the loop 210. Information from the probe 300 can be used, in some embodiments, to guide the reinsertion of the probe 300 through the loop 210.

As shown in FIG. 10S, the method can include narrowing the loop 210 on the distal end of the lasso wire 200 after the probe 300 has been inserted into and advanced through the loop 210 of the lasso wire 200. This can allow retraction across the IAS 1018. The lasso wire 200 and the guide catheter 100 can be retracted while the probe 300 remains in the left atrium 1016.

As shown in FIG. 10T, the method can include widening the loop 210. The probe 300 can be steered within the target area. In some embodiments, the guide catheter 100 and the lasso wire 200 can be retracted to a position in which a portion of the puncture needle 410 extends through the loop 210 of the lasso wire 200 prior to widening of the loop 210. In other embodiments, the loop 210 may be widened after the lasso wire 200 has been retracted through the IAS 1018 but before the loop 210 reaches the distal tip of the puncture needle 410.

In some embodiments in which the lasso wire 200 is entirely removable, the lasso wire 200 can be removed, leaving the puncture needle 410 in place. For example, this can be done by retracting one end of the lasso wire 200 from the proximal end of the guide catheter 100, such that the other end of the lasso wire 200 is pulled around the puncture needle guidewire 400, the puncture needle 410, and the probe 300, and back into and through the wire lumen of the guide catheter 100. In some embodiments, the guide catheter 100 can be removed prior to probe-guided intervention in the left atrium 1016.

As shown in FIG. 10V, the puncture needle 410 can be inserted into the left atrium 1016 and the probe 300 can be steered within the left atrium. After insertion, the user can proceed with probe-guided intervention in the left atrium 1016. Advantageously, in some embodiments, the probe 300 is in the left atrium 1016, and the puncture needle 410, or TSP, is in the left atrium 1016. Advantageously, in some embodiments, only a single point of insertion was used on the IAS 1018.

In some embodiments, a user can conduct an LAA closure procedure with the puncture needle 410 and the probe 300. In other embodiments, after the puncture needle 410 has been used to pierce the IAS 1018 or other anatomical boundary, the puncture needle can be exchanged for a separate LAA closure device. The separate LAA closure device can be inserted using the same introducer sheath as the puncture needle 410, which in some embodiments may be in a femoral vein not used for insertion of the guide catheter 100. The separate LAA closure device can be advanced into the left atrium 1016 using the same needle guidewire 400 used to advance the puncture needle 410. The LAA closure device may be delivered using a separate delivery catheter, which can traverse the single puncture site along with or along a path though the single puncture side substantially parallel to the probe 300. The LAA closure device can then be deployed from the delivery catheter during the LAA closure procedure.

In some embodiments, the probe 300 can traverse along a probe catheter, as discussed above. In some embodiments, advancement of the probe 300 can include advancement of a probe catheter relative to the guide catheter 100. In some embodiments, the lasso wire 200 can be retained within a wire sheath as discussed above. In some embodiments, advancement of the lasso wire 200 can include advancement of a wire sheath relative to the guide catheter 100.

Depending on the embodiment, certain acts, components, operations, or functions of any of the designs, configurations, processes, systems, or methods described herein can be performed or configured in a different sequence or configuration, may be added, merged, or left out altogether. Thus, in certain embodiments, not all described acts or components or operations or functions are necessary for the practice of the embodiment. No feature or group of features is necessary or essential for each embodiment.

What is claimed is:

1. A probe guide device for facilitating passage across an anatomical boundary, comprising:
a guide catheter comprising:
a first opening on a distal end of the guide catheter; and
a second opening on the distal end of the guide catheter;
a probe configured to be disposed within the guide catheter, wherein the probe is longitudinally translatable relative to the guide catheter to move a distal end of the probe out of the first opening on the distal end of the guide catheter; and
a retaining wire structure configured to be disposed within the guide catheter,
wherein the retaining wire structure comprises a looped portion at a distal end of the retaining wire structure,
wherein the retaining wire structure is configured to be longitudinally translatable relative to the guide catheter to be moved out of the second opening on the distal end of the guide catheter, and
wherein the probe is configured to be longitudinally translatable through the looped portion of the retaining wire structure when the looped portion of the retaining wire structure is positioned distal of the second opening on the distal end of the guide catheter.

2. The device of claim 1, wherein a width of the looped portion is adjustable from a proximal end of the retaining wire structure.

3. The device of claim 2, wherein the width of the looped portion is adjustable by partially retracting the retaining wire structure, wherein the looped portion is wider when the retaining wire structure is distally farther from the second opening on the distal end of the guide catheter.

4. The device of claim 1, wherein the distal end of the guide catheter comprises a dilator-tip extrusion.

5. The device of claim 1, wherein the guide catheter is steerable from a proximal end of the guide catheter.

6. The device of claim 1, further comprising a probe catheter configured to be disposed within the guide catheter, wherein the probe catheter comprises the probe.

7. The device of claim 4, wherein the probe catheter is steerable from a proximal end of the probe catheter.

8. The device of claim 1, further comprising a retaining wire sheath configured to be disposed within the guide catheter, and wherein the retaining wire structure is configured to be disposed within the retaining wire sheath.

9. The device of claim 1, wherein the looped portion is configured to be disengaged by retracting one end of the retaining wire structure from a proximal end of the retaining wire structure.

10. The device of claim 1, further comprising a removable obturator configured to fill the first opening on the distal end of the guide catheter.

11. A method for facilitating passage across an anatomical boundary, the method comprising:
positioning a guide catheter in an area near the anatomical boundary, wherein the guide catheter comprises:
a first opening on a distal end of the guide catheter; and
a second opening on the distal end of the guide catheter;
longitudinally translating a probe relative to the guide catheter so that a distal end of the probe extends through the first opening on the distal end of the guide catheter;
longitudinally translating a retaining wire structure relative to the guide catheter so that a distal end of the retaining wire structure extends through the second opening on the distal end of the guide catheter, wherein the retaining wire structure comprises a loop at a distal end of the retaining wire structure;
positioning a puncture needle within the loop in the area near the anatomical boundary, wherein the puncture needle comprises a puncture needle guidewire;
inserting the puncture needle across the anatomical boundary into a target area;
tracking the guide catheter across the anatomical boundary over the puncture needle guidewire into the target area; and
inserting the probe into the target area.

12. The method of claim 11, further comprising inserting the guide catheter in a vessel, and translating the guide catheter along the vessel to position the guide catheter in an area near the anatomical boundary.

13. The method of claim 11, wherein the anatomical boundary is an interatrial septum.

14. The method of claim 11, wherein the area near the anatomical boundary is a right atrium.

15. The method of claim 11, wherein the target area is a left atrium.

16. The method of claim 11, further comprising adjusting a width of the loop from a proximal end of the retaining wire structure.

17. The method of claim 11, further comprising disengaging the loop by retracting a first end of a wire forming a portion of the retaining wire structure.

18. The method of claim 13, further comprising puncturing the interatrial septum, to access a left atrium from a right atrium with an ICE probe and a medical device, via a single puncture site of the interatrial septum.

19. The method of claim 18, wherein the medical device comprises a left atrial appendage (LAA) closure device.

20. The method of claim 19, wherein the medical device further comprises a delivery catheter, at least a portion of the LAA closure device positioned within the delivery catheter when at least a portion of the medical device is advanced through the single puncture site of the interatrial septum to access the left atrium.

21. The method of claim 18, wherein the puncture needle is inserted into the area near the anatomical boundary via a first access site located in one of right or left femoral veins, and wherein the medical device is inserted into the area near the anatomical boundary via the same first access site as the puncture needle.

22. The method of claim 21, wherein the guide catheter is inserted into the area near the anatomical boundary via a second access site located in the other of the right or left femoral veins.
